(19) United States
(12) Patent Application Publication
(10) Pub. No.: US 2007/0072927 A1
(43) Pub. Date: Mar. 29, 2007

(54) NUTRITIONAL SUPPLEMENT FOR INCREASED ENERGY AND STAMINA

(76) Inventors: Joseph A. Vita, Boston, MA (US); Bruce N. Ames, Berkeley, CA (US); Benjamin V. Treadwell, Marco Island, FL (US)

Correspondence Address: THE LUTHER LAW FIRM, 12198 E. COLUMBINE DR., SCOTTSDALE, AZ, 85259, US

(21) Appl. No.: 11/463,881

(22) Filed: Aug. 10, 2006

Related U.S. Application Data
(63) Continuation-in-part of application No. 10/418,879, filed on Apr. 16, 2003, now abandoned.
Application No. 10/418,879 is a continuation-in-part of application No. 09/996,072, filed on Nov. 27, 2001, now Pat. No. 6,562,869.
Application No. 09/996,072 is a continuation-in-part of application No. 09/658,361, filed on Sep. 8, 2000, now Pat. No. 6,479,069.
(60) Provisional application No. 60/223,465, filed on Aug. 7, 2000.
(60) Provisional application No. 60/156,028, filed on Sep. 23, 1999.

(51) Int. Cl. A61K 31/4188; A61K 31/385; A61K 31/22; A61K 31/205

(52) U.S. Cl. 514/393; 514/440; 514/546; 514/554

(57) ABSTRACT

In a method of treating hypertension in individuals suffering therefrom, the method includes administering to said individuals an effective amount of carnitine and an effective amount of lipoic acid. A method of treating hypertension in individuals suffering therefrom includes administering twice a day a combination of 500 mg carnitine, 200 mg lipoic acid and optionally 100 micrograms of biotin. A method of treating hypertension in individuals with systolic blood pressure above about 135 mmHg includes administering a combination of carnitine, lipoic acid and optionally biotin.

### CROSS-REFERENCES TO RELATED APPLICATIONS

This application is a CIP of Ser. No. 10/418,879, filed Apr. 16, 2003, still pending, which is a CIP of Ser. No. 09/996,072, filed Nov. 27, 2001, now U.S. Pat. No. 6,562,869; which is a CIP of Ser. No. 09/658,361, filed Sep. 8, 2000, now U.S. Pat. No. 6,479,069; which claims the benefit of 60/156,028, filed Sep. 23, 1999, now expired; and the benefit of 60/223,465, filed Aug. 7, 2000, now expired.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant No. GL060886 awarded by the NIH.

### TECHNICAL FIELD

This invention is in the field of medical treatment, more specifically in the treatment of hypertension with a combination of lipoic acid and carnitine.

### BACKGROUND

Nutritional supplements are widely consumed by many people in the Western world. Some are of proven value, while others gradually lose favor, as no value is seen. Multivitamins are very popular among all age groups and are of known value. The new liquid diet supplements or nutritional drinks have been used to provide needed calories, protein, vitamins and minerals to people too sick or frail to eat sufficient amounts of nutrients.

Now these products are being marketed as energy boosters to people who want to remain energetic, particularly those aged 50 and older. The oldest, and by far the best selling, nutritional drinks are Ensure, Ensure Plus and Ensure Light made by the Ross Products Division of Abbott Laboratories (Columbus, Ohio). Sandoz Nutrition (Minneapolis, Minn.) sells ReSource to active older consumers. Mead Johnson Nutritionals (Evansville, Ind.) also has been marketing its Boost drink to seniors.

Registered dietitians state that these nutritional drinks are better than high calorie low-nutritional snacks, such as a bag of cheese curls and a soda. Although the nutritional drinks are being marketed as meal replacements, dietitians warn that the drinks are an inadequate substitute for three balanced meals. Each 8-ounce can or carton contains about 20-25% of the Recommended Daily Allowance of an assortment of vitamins and minerals but most lack fiber and other essential nutrients found in nature.

Recent research has suggested that taking sufficient quantities of certain substances rejuvenates aged intracellular bodies such as mitochondria, the failing powerhouses of cell metabolism. Numerous lines of evidence suggest that these organelles of cellular respiration, the especially the mitochondria, degenerate with cellular aging (Shigenaga et al. 1994, PNAS 91, 10771). Unfortunately, the study of mitochondrial aging has been hampered because mitochondria isolated from older cells and host animals are fragile and heterogeneous. Hence, the interpretation of any results has been suspect as about half the mitochondria generally lyse during isolation for unknown reasons. Recently, a new method was developed for studying mitochondria in hepatocytes from old animals that avoids this problem (Hagen et al. 1997, PNAS 94, 3064-3069). Mitochondria from older animals are not only more fragile, but have about half the level of cardiolipin, a key lipid unique to mitochondria, without which they cannot maintain a youthful high membrane potential. Furthermore, Hagen et al. show that in hepatocytes from older animals, the mitochondria have lower membrane potential and leak more toxic oxidants.

Carnitine and carnitine derivatives have been used as oral metabolic supplements in animal husbandry and for human diet and therapy. U.S. Pat. No. 5,362,753 (Method of increasing the hatchability of eggs by feeding hens carnitine); U.S. Pat. No. 4,687,782 (Nutritional composition for enhancing skeletal muscle adaptation to exercise training); U.S. Pat. No. 5,030,458 (Method for preventing diet-induced carnitine deficiency in domesticated dogs and cats); U.S. Pat. No. 5,030,657 (L-carnitine supplemented catfish diet); U.S. Pat. No. 4,343,816 (Pharmaceutical composition comprising an acyl-carnitine, for treating peripheral vascular diseases); U.S. Pat. No. 5,560,928 (Nutritional and/or dietary composition and method of using the same); U.S. Pat. No. 5,504,072 (Enteral nutritional composition having balanced amino acid profile); U.S. Pat. No. 5,391,550 (Compositions of matter and methods for increasing intracellular ATP levels and physical performance levels and for increasing the rate of wound repair); and U.S. Pat. No. 5,240,961 (Method of treating reduced insulin-like growth factor and bone loss associated with aging).

Similarly, mitochondrially active antioxidants including vitamins (especially C, E, B and D), glutathione, N-acetyl cysteine, lipoic acid, etc., have been used variously as human nutritional supplements and in dietary prophylaxis and therapy. For example, applications of lipoic acid have included U.S. Pat. No. 5,607,980 (Topical compositions having improved skin); U.S. Pat. No. 5,472,698 (Composition for enhancing lipid production in skin); U.S. Pat. No. 5,292,538 (Improved sustained energy and anabolic composition and method of making); U.S. Pat. No. 5,536,645 (Nutritive medium for the culture of microorganisms); and U.S. Pat. No. 5,326,699 (Serum-free medium for culturing animal cells).

Age-associated cellular bioenergetic degradation is gaining acceptance as the reason that the current human life expectancy is approximately 80 years, but life potential is estimated to be at least 120 years by certain experts. Bioenergetic degradation may contribute to various diseases of the aged, including heart failure, degenerative brain disease, muscle and vascular diseases, as well as other syndromes. A redox therapy based on coenzyme Q10 has been demonstrated to improve heart functions of old rats and not significantly affect those functions in young rats (Linnane A W, Kovalenki S and Gingold E B. Ann NY Acad Sci 854:202-13, 1998).

Coenzyme Q (or ubiquinone) plays a central role in the mitochondrial respiratory chain that uses energy for metabolism. It exists in mitochondria in the oxidized quinone form under aerobic conditions. In the reduced form ubiquinol, Q10 is an antioxidant. Q also is present in mitochondrial lipids. The structure of Q is very similar to those of the fat soluble vitamins A, D, K and E, which are all derived from isoprenoid structural units. Coenzyme Q10 has one polyisoprenoid side chain composed of ten isoprenoid units. Mitochondria need to maintain a large excess of Q, compared to other respiratory enzymes. Q is required to act on a mobile component of respiration that collects reducing equivalents from the more fixed complexes and passes them to other organelles and/or compounds.

Many conflicting reports have been published on the effectiveness of Q10 in various laboratory and clinical settings. Barbiroli et al. report that Q10 administration caused marked improvement in oxidative phosphorylation in both skeletal muscles and brains of patients with mitochondrial cytopathies due to enzyme defects (Biochimie 80(10): 847-53, 1998). On the other hand, Lass et al. studied the Q9 and Q10 content in brain, heart, skeletal muscle and other organs but found a decrease in mitochondrial Q9 and Q10 only in aging skeletal muscle (Biofactors 9(2-4): 199-205, 1999).

Life-long Q10 supplementation was studied in male rats and mice. Q10 did not prolong or shorten the lifespan of rats or mice. Plasma and liver levels were 2.6-8.4 times higher in the supplemented rats. Q10 levels in kidney, heart and brain were not affected by Q10 supplementation (Lonnrot K et al. Biochem Mol Biol Int 44(4):727-37, 1998).

To determine if Q10 has a neuroprotective effect, mice were first treated with Q10 or a control diet for four weeks. Then their striatal nerves were poisoned with 1-Me-4-Ph-1,2,3-tetrahydropyridine (MPTP). The mice continued on their assigned diets for another week before sacrifice. Both groups had considerable damage. However, the Q10-treated mice had 37% higher dopamine and 62% more dense neurons, indicating a protective effect of Q10 (Beal M F et al. Brain Res 783(1):109-14, 1998). Q10 also blocks the effects of doxorubicin, which stimulates mitochondrial oxidant production and a marked increase in mtDNA deletions in cardiac tissue (Adachi et al. Biochem Biophys Res Commun 195:945-51, 1993).

A group of healthy Finnish men and women aged 28-77 were tested for the total peroxyl radical trapping capacity of human plasma LDL phospholipids. There was an age-related difference in men, but not women. Most of the decrease occurred before age 50, remaining low into the 70's. Supplementation with Q10 doubled the peroxyl radical trapping capacity and thus may decrease LDL oxidation, which contributes to atherosclerosis (Aejmelaeus R et al. Mol Aspects Med 18 Suppl:S113-20, 1997).

Aging is very strongly associated with endothelial dysfunction and atherosclerosis. Moreover, blood pressure increases with age. The following are facts and conclusions from the Seventh Report of the Joint National Committee on Prevention, Detection, Evaluation and treatment of High Blood Pressure (Chobanian A V et al. Hypertension 2003 42:1206-1252). Beginning at 115/75 mmHg, cardiovascular disease risk doubles for each increment of 20/10 mmHg. More than half of people aged 60-69 have hypertension, and about 75% of those 70 or older are affected. Increasing blood pressure increases the stress on the endothelium and may exacerbate atherosclerosis. Systolic blood pressure (the higher of the two blood pressure numbers) over 140 mmHg is particularly deleterious. Two-thirds of patients with hypertension are not being controlled to systolic pressure less than 140 mmHg. The lowering of systolic blood pressure by 20 mmHg is expected to cut cardiovascular risk in half. Despite education efforts, approximately 30% of adults are still unaware of their hypertension, and more than 40% of individuals with hypertension are not on any treatment.

What is needed is a well tolerated treatment for treating hypertension, particularly that associated with metabolic syndrome and systolic blood pressure above 135 mmHg.

### SUMMARY OF INVENTION

The drawbacks and disadvantages of the prior art are overcome by the combination of carnitine and lipoic acid for hypertension.

In one embodiment, a method of treating hypertension in individuals suffering therefrom, in which the method includes administering to said individuals an effective amount of carnitine and an effective amount of lipoic acid. The combination of carnitine and lipoic acid can be administered daily. The carnitine can be administered in a quantity of about 0.025 grams/day to about 3 grams/day. The carnitine can be administered in a quantity of about 1 gram daily, which can be divided into two daily doses. The amount of lipoic acid is about 0.025 to about 1.5 grams daily. The lipoic acid can be administered in a quantity of about 0.4 grams per day, which can be divided into two daily doses. In addition, the method may include the administration of biotin, in a quantity of about 20 micrograms to about 1 milligram daily, preferably in a quantity of about 200 mcg per day, which can be delivered in two doses.

In another embodiment, there is a method of treating hypertension in individuals suffering therefrom calling for administering twice a day a combination of 500 mg carnitine and 200 mg lipoic acid. In addition, biotin can be administered, in an amount of about 20 mcg to about 1 mg daily, preferably about 200 mcg per day. The dose of biotin can be administered twice daily.

In yet another embodiment, there is a method of treating hypertension in individuals with systolic blood pressure above about 135 mmHg that includes administering a combination of carnitine and lipoic acid. The carnitine can be acetyl-L-carnitine. The lipoic acid can be R-α-lipoic acid. The combination of carnitine and lipoic acid can be administered daily. The carnitine can be administered in a quantity of about 0.025 grams/day to about 3 grams/day. The carnitine can be administered in a quantity of about 1 gram daily, which can be divided into two daily doses. The amount of lipoic acid can be about 0.025 to about 1.5 grams daily. The lipoic acid can be administered in a quantity of about 0.4 grams per day, which can be divided into two daily doses. In this method, the individual can be simultaneously treated with conventional anti-hypertensive medications. Simultaneously biotin can be given, in a quantity of about 20 mcg to 1 mg daily, preferably about 200 mcg per day, and preferably in two or more doses.

In yet another embodiment, there is a method of treating individuals with the metabolic syndrome that includes administering an effective amount of carnitine and an effective amount of lipoic acid. Optimally, the carnitine and lipoic acid are administered daily. The carnitine can be administered in a quantity of about 0.025 g/day to about 3 g/day, preferably about 1 g/day and preferably in two or more daily doses. The lipoic can be administered in a quantity of about 0.025 to about 1.5 g/day, preferably 0.4 g/day and preferably in two of more daily doses. Further, biotin also can be administered, preferably in a quantity of about 20 mcg/day to about 1 mg/day, more preferably about 200 mcg/day and preferably in two or more daily doses.

### DETAILED DESCRIPTION

Many older people lack two important basic nutrients: carnitine and lipoic acid. These two mitigate aging and provide more energy to older individuals and others with unhealthy mitochondria. Recent research has shown precisely how these two compounds work to promote healthy mitochondria, which power all cells. Mitochondria are responsible for the production of ATP and are present in relatively high numbers in essentially all cells of the body. The mitochondrial electron transport system consumes approximately 85% of the oxygen utilized by a cell. Cellular energy deficits caused by declines in mitochondrial function can impair normal cellular activities and compromise the cell's ability to adapt to various physiological stresses, a major factor in aging. Because of this high oxygen use, the mitochondria also have the highest production of deleterious oxidants.

Oxidants damage mitochondria in three important ways. Oxidants damage DNA, lipids and protein. The intra-mitochondrial DNA (mtDNA) have levels of oxidative damage which are at least 10-fold higher than those of nuclear DNA, which correlates with the 17-fold higher evolutionary mutation rate in mtDNA compared with nuclear DNA. mtDNA oxidation accumulates as a function of age, which has been shown in several species, including humans. This may lead to dysfunctional mitochondria. Mitochondrial protein damage is also age-related and may decrease energy production and increase oxidant production. Oxidative damage to mitochondrial lipids contributes to the decreasing fluidity of cell membranes with age. The lipid cardiolipin is a major component of the mitochondrial membrane and facilitates the activities of key mitochondrial inner membrane enzymes. The aged, damaged mitochondrial membrane cannot contain the oxidants, nor can it maintain as high a polarity as the younger membrane.

Fatty acid oxidation is an important energy source for many tissues. The activity of carnitine-acyl-carnitine exchange across the inner mitochondrial membrane is of great importance. The activity of this exchange reaction is decreased significantly with age, which may be due to a lower intra-mitochondrial pool of carnitine. L-carnitine or acetyl-L-carnitine has been shown to slow or reverse this age-related dysfunction. By itself, L-carnitine or acetyl-L-carnitine cannot correct the problem of excess oxidants. In fact, it was recently reported that carnitine supplementation increased oxidant production by 30% and decreased cell antioxidants markedly. Thus, acetyl-L-carnitine administration in older individuals may contribute to greater oxidative stress.

For the aged mitochondrial engines to run at maximum capacity, both carnitine and lipoic acid are essential. Lipoic acid is an antioxidant. And α-lipoic acid is a mitochondrial coenzyme that can help reverse the decline in metabolism seen with age. α-Lipoic acid supplementation has been shown to 1) reverse the age-related decrease in oxygen consumption, 2) restore the age-related decline in mitochondrial membrane potential, 3) triple the ambulatory activity of aged rats, 4) significantly lower the age-related increase in oxidants, and 5) restore glutathione and ascorbic acid levels to youthful levels.

Clearly, both carnitine and lipoic acid contribute to restoration of age-related mitochondria function and metabolic activity in older individuals. This contributes to improvements in energy, general health, mental acuity, immune system function, and skin and hair appearance and muscle mass.

Carnitine is available in many forms and all of those are included in the invention of the combination of carnitine and lipoic acid. Carnitine and carnitine derivatives have been used as metabolites in animal husbandry and for human diet and therapy. U.S. Pat. No. 5,362,753 (Method of increasing the hatchability of eggs by feeding hens carnitine); U.S. Pat. No. 4,687,782 (Nutritional composition for enhancing skeletal muscle adaptation to exercise training); U.S. Pat. No. 5,030,458 (Method for preventing diet-induced carnitine deficiency in domesticated dogs and cats); U.S. Pat. No. 5,030,657 (L-carnitine supplemented catfish diet); U.S. Pat. No. 4,343,816 (Pharmaceutical composition comprising an acyl-carnitine, for treating peripheral vascular diseases); U.S. Pat. No. 5,560,928 (Nutritional and/or dietary composition and method of using the same); U.S. Pat. No. 5,504,072 (Enteral nutritional composition having balanced amino acid profile); U.S. Pat. No. 5,391,550 (Compositions of matter and methods for increasing intracellular ATP levels and physical performance levels and for increasing the rate of wound repair); and U.S. Pat. No. 5,240,961 (Method of treating reduced insulin-like growth factor and bone loss associated with aging).

Most preferably, the carnitine is acetyl-L-carnitine. Acetyl-L-carnitine is preferred because it crosses the blood-brain barrier more readily, is more readily taken up by cells, can function as a donor of the acetyl group to choline to produce the neurotransmitter acetylcholine, and is more effective than L-carnitine in neuroprotection. It also can reverse the age-related decrease in cardiolipin, age-associated decrease in mtDNA transcription, and decreased membrane potential.

Generally, a daily dosage of carnitine is about 10 milligrams per day (mg/day) to about 8 grams per day (g/day). Preferably, the amount of carnitine in the composition is about 25 mg to about 1,500 mg (or about 0.025 g to about 1.5 g). More preferably, the amount of carnitine given per day is about 1000 mg (or about 1 g) per day. Most preferably, the amount of carnitine in the composition (administered twice a day) is about 500 mg (or about 0.5 g).

Lipoic acid (or thioctic acid) is a mitochondrially active antioxidant that physiologically comprises a metabolically reactive thiol group. Mitochondrially active antioxidants including certain vitamins (especially vitamins C, E, B and D), glutathione, N-acetyl cysteine (NAC), lipoic acid, their derivatives, etc., have been used variously as human nutritional supplements and in dietary prophylaxis and therapy. For example, applications of lipoic acid have included U.S. Pat. No. 5,607,980 (Topical compositions having improved skin); U.S. Pat. No. 5,472,698 (Composition for enhancing lipid production in skin); U.S. Pat. No. 5,292,538 (Improved sustained energy and anabolic composition and method of making); U.S. Pat. No. 5,536,645 (Nutritive medium for the culture of microorganisms); and U.S. Pat. No. 5,326,699 (Serum-free medium for culturing animal cells). Preferably, the compound is at least one of glutathione, N-acetyl cysteine and lipoic acid. Metabolites of lipoic acid have been found to have a longer half-life and also are suitable for supplementation.

When large amounts of free alpha-lipoic acid are available, alpha-lipoic is also able to function as an antioxidant. Alpha-dihydrolipoic acid (DHLA) is the reduced form of alpha-lipoic acid and generally is the only form that functions directly as an antioxidant. Free alpha-lipoic acid is rapidly taken up by cells and reduced to DHLA intracellularly. DHLA may prevent oxidative damage by interacting with potentially damaging reactive oxygen species (ROS) and reactive nitrogen species (RNS). DHLA also regenerates other antioxidants which become oxidized when they neutralize free radicals. Specifically DHLA can reduce oxidized vitamin C, glutathione and coenzyme Q10, which in turn regenerates vitamin E, forming an antioxidant network. DHLA may help regulate the transcription of certain genes involved in inflammation and pathology of a number of diseases, including atherosclerosis. Oxidative stress has been implicated in the pathology of diabetic neuropathy, and alpha-lipoic acid is approved for its treatment in Germany. Alpha-lipoic acid may also protect against heart disease and cancer. Alpha-lipoic acid also protects against cholesterol oxidation and the consequent atherosclerosis in individuals at risk of cardiovascular disease.

Lipoic acid is the antioxidant of choice with acetyl-L-carnitine because it functions in concert with acetyl-L-carnitine in energy production and the synthesis of acetylcholine. Lipoic acid and acetyl-L-carnitine are produced by the cell and are present in the mitochondria. Lipoic acid is also one of the more potent natural antioxidants present in the body that has the ability to protect both lipid and water-soluble components. Manufactured alpha-lipoic acid occurs in a racemic form comprising almost equal amounts of the D- and L-forms. Although only R-alpha-lipoic acid occurs naturally, s-alpha-lipoic acid is reduced to DHLA as R-alpha-lipoic acid in the cytosol (protein-rich fluid of cells).

A daily dosage of racemic lipoic acid is generally about 10 mg/day to about 8 g/day. Preferably, the amount of lipoic acid in the composition is about 25 mg to about 1,500 mg (or about 0.025 g to about 1.5 g). More preferably, the amount of lipoic acid in the composition is about 40 mg to about 700 mg (or about 0.040 g to about 0.7 g). Most preferably, the amount of lipoic acid ingested per day is about 400 mg (or 0.4 g). Divided into two doses, each provides about 200 mg (or about 0.2 g). The dose can be provided in a variety of dosage forms, most preferably tablets or capsules. If provided as R-lipoic acid, it is hypothesized that the composition need only contain one half of the racemic mixture, including 200 mg/day and 100 mg per dose.

An absence of vitamins in the human diet can lead to illness and disease. B complex vitamins have proven to be essential for human nutrition. The vitamin B complex comprises a large number of compounds. Traditional members of the vitamin B complex include thiamine, riboflavin, nicotinic acid, pyridoxine, pantothenic acid, biotin, folic acid, cyanocobalamin, choline, inositol and para-amino-benzoic acid

Because of their water solubility, any excesses of B vitamins are not stored but are excreted in the urine. Therefore, it is rare that B vitamins, except for very high levels of B6 and folate, would accumulate in the human body to toxic levels. For the same reason, the storage of B vitamins in the body is limited and therefore, B vitamins must be ingested regularly. The composition of the present invention contains at least one component of the B vitamin complex.

Biotin is an important nutrient required in a number of biochemical reactions, including those involving fat and carbohydrate metabolism. It is important for maintaining optimal levels of metabolites utilized for energy production in the mitochondria. Biotin (formerly known as Vitamin H and W Factor) is classified as a component of the Vitamin B₂ Complex and is chemically defined as cis-hexahydro-2-oxo-1H-thieno[3,4-d]imidazoline-4-valeric acid. Biotin is a bicyclic compound, of which the tetrahydrothiophene ring contains sulfur, and has the β-valeric acid side chain. The second ring contains a ureido group. The molecule exists in eight (8) stereoisomeric (enantiomeric) forms, only one of which is biologically active in humans (supra vida). Biotin is an essential micronutrient for many organisms, including humans, is inactivated by native, uncooked avidin (an enzyme present in raw egg whites), and probably by beta-oxidation of the beta side chain. The chemical structure of biotin is shown in Fig. xxxxx. Biotin, an essential micronutrient in human nutrition, is present in relatively low amounts, compared to other B vitamins. However, biotin cannot be synthesized by mammals, and humans are dependent on the synthetic capabilities of microflora (e.g., bacteria) in the human intestine to contribute to the human need for this vitamin. Biotin is extremely important metabolically as it plays essential roles in the biosynthesis of such macronutrients as fatty acids, gluconeogenesis, metabolism of critical branched-chain amino acids (e.g., L-leucine, L-isoleucine, and L-valine). Biotin also is integral to the de novo synthesis of purine nucleosides and participates in gene expression at both the transcriptional and translational phases, and may participate in the replication of DNA as well.

Dietary sources with the assistance of microflora in the human gastrointestinal tract generally provide adequate amounts of biotin. However, biotin deficiency, while rare, does occur. Common causes include total parenteral nutrition, malabsorption syndromes, without adequate biotin supplementation, and antibiotic therapy, killing microflora in the large intestine. Biotin deficiency has been linked to teratogenicity and occurs in both neonates and adults. Of special concern is the fact that even marginal biotin deficiency during pregnancy may result in deleterious effects. Clinical signs and symptoms of biotin deficiency include, but are not limited to, the skin and its appendages, such as hair, eyes, unusual fat distribution, neurological disorders, and developmental failure in neonates. Neonatal biotin deficiency frequently is the result of functional inborn errors of metabolism (e.g., carboxylase deficiency, holocarboxylase synthetase deficiency, biotinidase deficiency, and proprionyl-CoA carboxylase deficiency). Recent evidence indicates that biotin plays a role in glucose and lipid metabolism. Therefore, biotin-induced improvement in glucose metabolism could play a significant role in the management of diabetes mellitus, especially those patients afflicted with non-insulin dependent diabetes mellitus, insulin resistance and/or the metabolic syndrome at the cellular level.

The molecular structure of biotin is similar to that of α-lipoic acid. As a consequence of this structural similarity, an excess of α-lipoic acid could compete with or displace biotin from its enzymes, according to experimental evidence from cell and animal studies. For this reason, biotin has been added to the formulation to offset a potential imbalance with the above-mentioned lipoic acid.

The Recommended Daily Allowance for biotin from the United States government is 300 micrograms. A daily dosage of biotin can be about 2 microgram/day to about 1 mg/day. Preferably, the amount of biotin in the composition is about 20 microgram to about 500 micrograms per day. More preferably, the amount of biotin administered with lipoic acid is about 200 micrograms per day. Divided into two pills administered daily, each composition contains about 100 micrograms of biotin.

The metabolic syndrome is also known as metabolic syndrome X, syndrome X, insulin-resistance syndrome, and dysmetabolic syndrome. The metabolic syndrome is a common clinical condition that affects 20 to 25 percent or more of the general population of the United States and the prevalence of this syndrome increases with age, with a prevalence approaching 40 percent or more of individuals in the seventh and eighth decades of life. The incidence of the metabolic syndrome appears to be increasing. The metabolic syndrome is associated with cardiovascular disease, obesity, and diabetes mellitus.

The constellation of the following factors characterizes the metabolic syndrome when fully expressed and manifest:
Abdominal obesity (also known as visceral obesity, central obesity, or hypertrigylceridemic waist), a condition known to be associated with cardiovascular disease;
Dyslipidemias, including hypertrigyceridemia, high levels of low-density lipoproteinemia (LDL), and low levels of high density lipoproteinemia (HDL) that are known risk factors in the development of atherosclerosis and cardiovascular disease;
Hypertension, a risk factor for cardiovascular disease;
Diabetes mellitus, especially insulin resistance or type II diabetes mellitus;
Hyperthrombotic state, or prothrombotic state (e.g., associated with high circulating levels of fibrinogen or plasminogen activator inhibitor-1); and
Proinflammatory propensity (e.g., elevated circulating C-reactive protein).

The metabolic syndrome is associated with increased levels of angiotension II activity (associated with essential arterial hypertension), induction of proinflammatory and oxidative states, as indicated above, and endothelial dysfunction. Lipoic acid, a known antioxidant, is believed to affect endothelial function and inflammatory responses in patients with the metabolic syndrome, and thus is of potential therapeutic value in patients with this diagnosis.

Individuals with the metabolic syndrome as described above are at significantly higher risk of developing overt manifestations of atherosclerosis, suggesting that abnormal endothelial function may be a significant factor in the metabolic syndrome, including the coronary arteries (angina pectoris and acute myocardial infarction), cerebral arteries (cerebrovascular accidents or strokes), and peripheral artery disease (claudication), as well as diabetes mellitus type II, and all of the known sequellae of this disease.

The molecular biology and pathophysiology of the metabolic syndrome remain elusive and are most likely complex and multifactorial in nature. The underlying risk factors for the metabolic syndrome have not been identified with clarity. Abdominal or visceral obesity, insulin resistance at the cellular level, physical inactivity, aging, undefined hormonal abnormalities, and genetic predisposition have been proposed as contributing factors to the development of the metabolic syndrome and its serious clinical manifestations. Abdominal obesity and insulin resistance appear to be the dominant contributing factors to the metabolic syndrome. Some individuals are predisposed genetically to develop the metabolic syndrome and insulin resistance, whereas other acquired factors, such as physical inactivity and development of abdominal/visceral obesity, are either manifestations of the metabolic syndrome or are contributing factors to the syndrome. The association between obesity and insulin resistance is well known.

The metabolic syndrome is very common in individuals who present with arterial hypertension, diabetes mellitus, and obesity. The exact criteria for establishing the diagnosis of the metabolic syndrome are not uniformly accepted. However, the National Cholesterol Education Program's Adult Treatment Panel III recommendations are currently widely used, with some modifications. The American Heart Association and the National Heart, Lung, and Blood Institute currently recommend that the metabolic syndrome should be diagnosed by the presence of at least three of the following factors:
Excessive waist circumference (≧40 inches or 102 cm for men; 35 inches or 88 cm for women);
Hypertriglyceridemia (e.g., ≧150 mg/dL, fasting);
Reduced HDL (<40 mg/dL for men; ≦50 mg/dL for women, fasting);
Arterial hypertension (≧approximately 135/85 mm Hg for both genders); and
Hyperglycemia (recently redefined as ≧100 mg/dL, fasting).

The American Heart Association recommends several treatments for the management of metabolic syndrome. First, the primary goal is to reduce the risks of developing cardiovascular disease and diabetes type II and the associated complications of these serious medical conditions. Those skilled in the art of medicine would recommend, as first-line therapeutic measures: stop smoking tobacco, reduce LDL cholesterol and increase HDL cholesterol, manage arterial hypertension to achieve acceptable levels (no consensus on therapeutically-acceptable levels of systolic or diastolic blood pressure exist, but ≦135/85 mm Hg appears to be reasonable, although lower blood pressures may provide additional health benefits), and, finally, to reduce blood glucose levels as currently recommended to ≦110 mg/dL or, preferably, ≦100 mg/dL. In addition, life-style changes are generally recommended and include increased physical activity (mild to moderate activity on most days of the week), body weight reduction via a balanced program of increased physical activity and reduced caloric intake with the goal of achieving a body mass index (BMI) of <25 kg/m², and a diet that reduces consumption of total calories, cholesterol, saturated fat, and certain “trans” fats.

Recently published data strongly suggest that the administration of lipoic acid and irbesartan reduce markers of inflammation in the metabolic syndrome (cf., Sola, Circ). The authors conclude that administration of irbesartan and/or lipoic acid to patients with metabolic syndrome improves endothelial function and reduces proinflammatory markers. These factors are known to be implicated in the pathogenesis of atherosclerosis, and the subsequent consequences of this deleterious and insidious pathological process.

Coenzyme Q10 (Q10) is an important supplement. In groups of males and females ranging from 90-106 years, the prevalence of inadequate Q10 status was 40% for women and 24% for men. In women, the decreased Q10 was associated with impaired natural killer cell effectiveness (p<0.05), indicating decreased abilities to fight infections and to quickly eliminate individual cancer cells as they first develop. Q10 also appears to block programmed cell death, or apoptosis, through its action in the mitochondria (Kagan T et al., Ann NY Acad Sci 887:31-47, 1999). Furthermore, Q10, in its reduced form of ubiquinol-10 that is normally present in the blood, appears to protect human lymphocytes from oxidative damage to their DNA (Tomasetti et al., Free Radic Biol Med 27 (9-10):1027-32, November 1999). No important adverse effects have been reported from human studies using daily supplements of up to 200 mg Q10 for 6 to 12 months and 100 mg daily for up to 6 years. (See Overvad K et al. Eur J Clin Nutr 53(10):764-70, 1999).

Q10 also may contribute to anti-aging effect by protecting against atherosclerosis that also results from oxidative stress. (See Pedersen H S, et al., Biofactors 9(2-4): 319-23, 1999). Q10 also improves the tolerance of the senescent myocardium to aerobic and ischemic stress in human atrial tissue and rats. Q10 corrected the age-specific, diminished recovery of function in older hearts so that older hearts recovered function at a similar rate to younger ones (See Rosenfeldt F L et al. Biofactors 9(2-4): 291-9, 1999).

As for the supplemental dose of Q10, older Finnish men obtained benefit from 100 mg/day. A woman deficient in Q10 received 150 mg/kg and rapidly improved (Sobriera et al. Neurology 48:1283-43, 1997). Q10 has also been used at dose of about 200 mg/day to help improve heart function in persons with hypertrophic cardiomyopathy.

Based on this information, a supplemental dosage of Q10 can optionally be added to the composition. A preferred amount of Q10 added to the composition is about 20 mg to about 250 mg (or about 0.020 g to about 0.25 g). More preferably, the amount of Q10 in the composition is about 100 mg.

Additional nutrients are important in older individuals, including but not limited to calcium, vitamin D, other B vitamins, Vitamins C and/or E, iron and zinc. Many of these nutrients have been found to be deficient in the diets of elders and thus can be appropriately supplemented with multivitamins or other preparations.

A preferred formulation provides lipoic acid, carnitine, and optionally in combination with biotin and Q10 in a timed release formulation to provide a steady supply of the nutrients to the mitochondria which work 24 hours a day. One method of accomplishing timed release is chemically combining the micronutrient(s) with other nutrients, salts (as determined by those skilled in the pharmaceutical art), which generally slows the process of making the micronutrient(s) available. The use of different salts, counter-ions, etc. of the micronutrients with different dissolution rates provides for the desired gradual release of each micronutrient.

Besides these methods, two other basic systems are used to control chemical release: coating a core comprising the micronutrient(s) and excipients (coated system) and incorporating the micronutrient(s) into a matrix (matrix system). Coated systems involve the preparation of product-loaded cores coated with release rate-retarding materials. Product-loaded cores can be formulated as microspheres, granules, pellets or core tablets. There are many known core preparation methods, including, but not limited to, 1) producing granules by top-sprayed, fluidized-bed granulation, or by solution/suspension/powdering layering by Wurster coating; 2) producing spherical granules or pellets by extrusion-spheronization, rotary processing, and melt pelletization; 3) producing core tablets by compression and coating with a release rate-retarding material; and 4) producing microspheres by emulsification and spray-drying.

Matrix systems embed the micronutrient in a slowly disintegrating or slow-release matrix. Rate of release is controlled by the erosion of the matrix and/or by the diffusion of the micronutrient(s) through the matrix. In general, the active product substance, excipients and the release rate-retarding materials are mixed and then processed into matrix pellets or tablets. Matrix pellets can be formed by granulation, spheronization using cellulosic materials, or by melt pelletization using release retardant materials, while matrix tablets are prepared by compression in a tablet press. An example of a cellulosic material is hydroxypropyl-methylcellulose as a release-rate retarding material.

Coated or matrix formulations can be filled into capsules, compression formulations, tablets or other formulations. The rate of release can be further modified by those skilled in the art to obtain the desired product release profile. Pellets containing any of lipoic acid, carnitine, biotin, or optionally other B vitamins and Q10 can be blended to form a combination product.

Convenient assays for the requisite bioactivities are described above or in the references cited herein. For example, cardiolipin content is readily assayed as referenced in Guan, Z. Z., Soderberg, M., Sindelar, P., and Edlund, C. Content and Fatty Acid Composition of Cardiolipin in the Brain of Patients with Alzheimer's Disease. Neurochem. Int. 25: 295-300, 1994. Oxidant production (DCFH) may be assayed as described by LeBel, C. P., Ischiropoulos, H., and Bondy, S. C. Evaluation of the Probe 2′,7′-Dichlorofluorescin as an Indicator of Reactive Oxygen Species Formation and Oxidative Stress. Chem. Res. Toxicol. 5: 227-231, 1992. Assays for parameters of aging such as host activity and behavior such as grooming, sexual activity, dominance, coat condition, wound repair, including molecular lesions, muscle strength and tone, kidney appearance and function, etc. are similarly well known in the art.

### EXAMPLE

A double blinded, randomized, placebo-controlled crossover study was performed to examine the effects of a combination of acetyl-L-carnitine and α-lipoic acid in older patients with cardiovascular disease. Each individual underwent an 8-week treatment period with either active treatment or placebo, a 4-week washout period and a second 8-week period with alternative treatment.

The older patients (at least 55 years old) had proven coronary artery disease confirmed by coronary angiography or a history of myocardial infarction (as confirmed by ECG changes and elevation of serum creatine kinase or troponin I). Other therapies to reduce cardiovascular risk were continued, including lipid-lowering therapy, aspirin and angiotensin converting enzyme (ACE) inhibitors.

The study used a combination capsule containing 500 mg of acetyl-L-carnitine and 200 mg of α-lipoic acid or matching placebo capsules. Patients were telephoned at 4-week intervals to determine compliance with the regimen. Prior to each study visit, patients were asked to fast, refrain from smoking overnight and stop all vasoactive medications for 24 hr (including nitrates, calcium channel blockers, ACE inhibitors, β-adrenergic blockers and others).

On the day of the study visit, a blood sample was collected, blood pressure was determined using an automatic cuff, and endothelium-dependent flow-mediated dilation of the brachial artery was assessed by ultrasound. The vasodilator response to sublingually administered nitroglycerin (0.4 mg) was determined to evaluate the effect of therapy on non-endothelium-dependent vasodilation to evaluate the function of vascular smooth muscle.

The blood sample was processed for plasma lipid profile and the plasma levels of α-lipoic acid and acetyl-L-carnitine to confirm compliance. The mitochondrial membrane potential of white blood cells was tested as an indication of whether mitochondrial function was in fact improved.

There was no treatment effect on brachial artery flow-mediated dilation, reactive hyperemia or nitroglycerin-mediated dilation. A trend for reduced systolic blood pressure and pulse pressure after active treatment was observed for the group as a whole. The following blood pressure comparisons are drawn from values measured before and after the active, treatment phase of the study. A significant reduction in blood pressure occurred in the patients with blood pressure above the median (≧135 mmHg), from an average of 151±20 mmHg to 142±18 mmHg, p=0.03 by repeated measures ANOVA. In addition, the pulse pressure significantly decreased from 73±16 mmHg to 68±15 mmHg, p=0.03. In the subgroups with metabolic syndrome or Type 2 diabetes, both of which contribute to heart disease, there also was a significant decrease in blood pressure from an average of 139±21 mmHg to 130±18 mmHg, p=0.03. In addition, the pulse pressure significantly decreased from 63±18 mmHg to 60±15 mmHg, p=0.04.

Because it has been determined that increasing the blood pressure by 20/10 mmHg doubles the cardiovascular risk and the tested individuals described herein were already optimally treated with conventional antihypertensive medication(s), it is believed that the 9 mmHg decrease associated with treatment with the carnitine-lipoic acid combination in blood pressure contributes to improved health.

All patents and patent publications cited in this specification are herein incorporated by reference as if each individual patent or patent publication were specifically, and individually, indicated to be incorporated by reference.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed as the best mode contemplated for carrying out this invention, but that the invention will include all embodiments falling within the scope of the appended claims.

1. A method of treating hypertension in individuals suffering therefrom, the method comprising administering to said individuals an effective amount of carnitine and an effective amount of lipoic acid.

2. The method of claim 1 wherein the combination of carnitine and lipoic acid is administered daily.

3. The method of claim 1 wherein the carnitine is administered in a quantity of about 0.025 grams/day to about 3 grams/day.

4. The method of claim 1 wherein the carnitine is administered in a quantity of about 1 gram daily.

5. The method of claim 4 wherein the carnitine is divided into two daily doses.

6. The method of claim 1 wherein the amount of lipoic acid is about 0.025 to about 1.5 grams daily.

7. The method of claim 1 wherein the lipoic acid is administered in a quantity of about 0.4 grams per day.

8. The method of claim 7 wherein lipoic acid is divided into two daily doses.

9. The method of claim 1 further comprising administering biotin.

10. The method of claim 9 wherein the biotin is administered in a quantity of about 20 micrograms to 1 milligram daily.

11. The method of claim 9 wherein the biotin is administered in a quantity of about 200 mcg per day.

12. The method of claim 11 wherein the 200 mcg of biotin is administered in two or more doses.

13. A method of treating hypertension in individuals suffering therefrom comprising administering a combination of 500 mg carnitine and 200 mg lipoic acid in one or more daily doses.

14. The method of claim 13 further comprising administering biotin.

15. The method of claim 14 wherein the biotin is administered in a quantity of about 20 micrograms to 1 milligram daily.

16. The method of claim 14 wherein the biotin is administered in a quantity of about 200 mcg per day.

17. The method of claim 16 wherein the 200 mcg of biotin is administered in two or more doses.

18. A method of treating hypertension in individuals with systolic blood pressure above about 135 mmHg comprising administering a combination of carnitine and lipoic acid.

19. The method of claim 18 wherein the carnitine is acetyl-L-carnitine.

20. The method of claim 18 wherein the lipoic acid is R-α-lipoic acid.

21. The method of claim 18 wherein the combination of carnitine and lipoic acid is administered daily.

22. The method of claim 18 wherein the carnitine is administered in a quantity of about 0.025 grams/day to about 3 grams/day.

23. The method of claim 18 wherein the carnitine is administered in a quantity of about 1 gram daily.

24. The method of claim 15 wherein the carnitine is divided into two daily doses.

25. The method of claim 18 wherein the amount of lipoic acid is about 0.025 to about 1.5 grams daily.

26. The method of claim 18 wherein the lipoic acid is administered in a quantity of about 0.4 grams per day.

27. The method of claim 18 wherein lipoic acid is divided into two or more daily doses.

28. The method of claim 18, wherein the individual is simultaneously treated with conventional anti-hypertensive medications.

29. The method of claim 18 further comprising administering biotin.

30. The method of claim 21 wherein the biotin is administered in a quantity of about 50 micrograms to 1 milligram daily.

31. The method of claim 21 wherein the biotin is administered in a quantity of about 200 mcg per day.

32. The method of claim 23 wherein the 200 mcg of biotin is administered in two or more doses.

33. A method of treating individuals with the metabolic syndrome, the method comprising administering thereto an effective amount of carnitine and an effective amount of lipoic acid.

34. The method of claim 33 wherein the combination of carnitine and lipoic acid is administered daily.

35. The method of claim 33 wherein the carnitine is administered in a quantity of about 0.025 grams/day to about 3 grams/day.

36. The method of claim 33 wherein the carnitine is administered in a quantity of about 1 gram daily.

37. The method of claim 33 wherein the carnitine is divided into two daily doses.

38. The method of claim 33 wherein the amount of lipoic acid is about 0.025 to about 1.5 grams daily.

39. The method of claim 33 wherein the lipoic acid is administered in a quantity of about 0.4 grams per day.

40. The method of claim 39 wherein lipoic acid is divided into two daily doses.

41. The method of claim 33 further comprising administering biotin.

42. The method of claim 41 wherein the biotin is administered in a quantity of about 20 micrograms to 1 milligram daily.

43. The method of claim 42 wherein the biotin is administered in a quantity of about 200 mcg per day.

44. The method of claim 43 wherein the 200 mcg of biotin is administered in two or more doses.
